(19) United States
(12) Patent Application Publication
(10) Pub. No.: US 2006/0190003 A1
(43) Pub. Date: Aug. 24, 2006

(54) Surgical method

(75) Inventors: Mikhail Boukhny, Laguna Niguel, CA (US); James Y. Chon, Irvine, CA (US); Bruno Dacquay, Irvine, CA (US)

Correspondence Address: ALCON, IP LEGAL, TB4-8, 6201 SOUTH FREEWAY, FORT WORTH, TX, 76134, US

(73) Assignee: ALCON, INC.

(21) Appl. No.: 11/232,205

(22) Filed: Sep. 21, 2005

Related U.S. Application Data
(63) Continuation-in-part of application No. 11/060,827, filed on Feb. 18, 2005.

(51) Int. Cl. A61B 17/20

(52) U.S. Cl. 606/107; 604/22

(57) ABSTRACT

A surgical method using a bent phacoemulsification tip and vibrating the tip torsionally so as to produce a whipping motion in the distal end of the tip.

This application is a continuation -in-part of U.S. patent application Ser. No. 11/060,827, filed Feb. 18, 2005, currently co-pending.

### BACKGROUND OF THE INVENTION

This invention relates generally to the field of phacoemulsification and more particularly to torsional phacoemulsification cutting tips.

The human eye in its simplest terms functions to provide vision by transmitting light through a clear outer portion called the cornea, and focusing the image by way of the lens onto the retina. The quality of the focused image depends on many factors including the size and shape of the eye, and the transparency of the cornea and lens.

When age or disease causes the lens to become less transparent, vision deteriorates because of the diminished light which can be transmitted to the retina. This deficiency in the lens of the eye is medically known as a cataract. An accepted treatment for this condition is surgical removal of the lens and replacement of the lens function by an IOL.

In the United States, the majority of cataractous lenses are removed by a surgical technique called phacoemulsification. During this procedure, a thin phacoemulsification cutting tip is inserted into the diseased lens and vibrated ultrasonically. The vibrating cutting tip liquifies or emulsifies the lens so that the lens may be aspirated out of the eye. The diseased lens, once removed, is replaced by an artificial lens.

A typical ultrasonic surgical device suitable for ophthalmic procedures consists of an ultrasonically driven handpiece, an attached cutting tip, and irrigating sleeve and an electronic control console. The handpiece assembly is attached to the control console by an electric cable and flexible tubings. Through the electric cable, the console varies the power level transmitted by the handpiece to the attached cutting tip and the flexible tubings supply irrigation fluid to and draw aspiration fluid from the eye through the handpiece assembly.

The operative part of the handpiece is a centrally located, hollow resonating bar or horn directly attached to a set of piezoelectric crystals. The crystals supply the required ultrasonic vibration needed to drive both the horn and the attached cutting tip during phacoemulsification and are controlled by the console. The crystal/horn assembly is suspended within the hollow body or shell of the handpiece by flexible mountings. The handpiece body terminates in a reduced diameter portion or nosecone at the body's distal end. The nosecone is externally threaded to accept the irrigation sleeve. Likewise, the horn bore is internally threaded at its distal end to receive the external threads of the cutting tip. The irrigation sleeve also has an internally threaded bore that is screwed onto the external threads of the nosecone. The cutting tip is adjusted so that the tip projects only a predetermined amount past the open end of the irrigating sleeve. Ultrasonic handpieces and cutting tips are more fully described in U.S. Pat. Nos. 3,589,363; 4,223,676; 4, 246, 902; 4,493,694; 4,515,583; 4,589,415; 4,609,368; 4,869,715; 4,922,902; 4,989,583; 5,154,694 and 5,359,996, the entire contents of which are incorporated herein by reference.

In use, the ends of the cutting tip and irrigating sleeve are inserted into a small incision of predetermined width in the cornea, sclera, or other location. The cutting tip is ultrasonically vibrated along its longitudinal axis within the irrigating sleeve by the crystal-driven ultrasonic horn, thereby emulsifying the selected tissue in situ. The hollow bore of the cutting tip communicates with the bore in the horn that in turn communicates with the aspiration line from the handpiece to the console. A reduced pressure or vacuum source in the console draws or aspirates the emulsified tissue from the eye through the open end of the cutting tip, the cutting tip and horn bores and the aspiration line and into a collection device. The aspiration of emulsified tissue is aided by a saline flushing solution or irrigant that is injected into the surgical site through the small annular gap between the inside surface of the irrigating sleeve and the cutting tip.

One phacoemulsification tip that has gained widespread acceptance has a belled or flared distal end. Such a tip is described in U.S. Pat. No. 4,816,018 (Parisi). Such a design allows for larger lens material purchase as well as increased holding force when vacuum is applied to the tip while maintaining a smaller bore in the shaft of the tip. This combination of features increases anterior chamber stability, by reducing sudden outflow from the anterior chamber when the distal end becomes occluded and this occlusion breaks.

Another phacoemulsification tip is an angled or “bent” tip with or without a flared distal end. These tips are described in U.S. Pat. No. 6,039,715 (Mackool), U.S. Pat. No. 5,653,724 (Imonti) and U.S. Pat. No. 5,154,694 (Kelman). These tips have a predominantly straight shaft with the far distal portion of the shaft being bent on an angle. Bent tips are used by a great many surgeons, and are particularly useful when used in conjunction with a oscillatory phacoemulsification handpiece, such as those described in U.S. Pat. No. 6,352,519 (Anis, et al.) and U.S. Pat. No. 6,602,193 (Chon) and commercially available as the NeoSoniX® handpiece from Alcon Laboratories, Inc., Fort Worth, Tex., however; when used with these handpieces, bent tips still required some longitudinal movement in addition to oscillatory movement.

The inventors have discovered that angled phacoemulsification tip are particularly advantageous when used in combination with torsional ultrasound handpiece. Torsional ultrasound handpieces are more fully disclosed in U.S. Pat. No. 6,077,285 (Boukhny) and other pending applications. Therefore, a need continues to exist for an angled phacoemulsification tip that cuts more efficiently during torsional vibration of the tip.

### BRIEF SUMMARY OF THE INVENTION

The present invention improves upon the prior art by providing a surgical method using a bent phacoemulsification tip and vibrating the tip torsionally so as to produce a whipping motion in the distal end of the tip.

Accordingly, one objective of the present invention is to provide a method of vibrating a phacoemulsification cutting tip with increased efficiency, particularly during torsional ultrasound movement.

Another objective of the present invention is to provide a method of vibrating a phacoemulsification cutting tip having a bent tip.

These and other advantages and objectives of the present invention will become apparent from the detailed description and claims that follow.

### BRIEF DESCRIPTION OF THE DRAWINGS

FIG. 1 is a perspective view of a handpiece and control console that may be used with the present invention.

FIG. 2 is a perspective view of the distal end of a typical prior art straight shaft phacoemulsification tip.

FIG. 3 is an elevational view the distal end of a typical prior art angled or bent phacoemulsification tip.

### DETAILED DESCRIPTION OF THE INVENTION

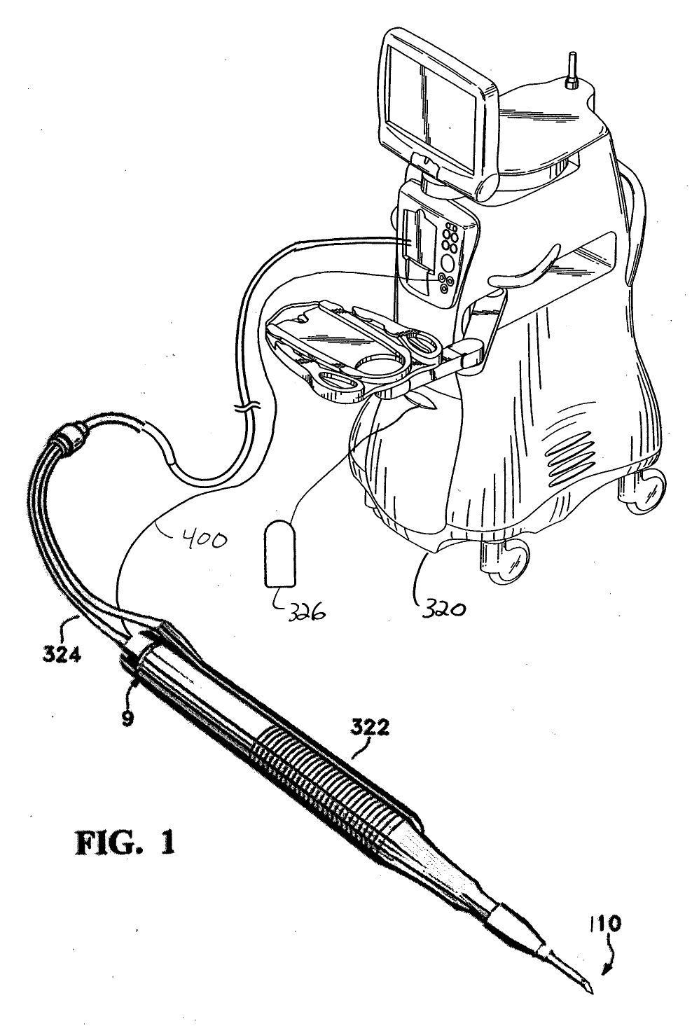

As best seen in FIG. 1, surgical console 320 suitable for use with the present invention may be any commercially available surgical control console such as the INFINITI® surgical systems available from Alcon Laboratories, Inc., Fort Worth, Tex. Console 320 is connected to handpiece 9 through irrigation line 322 and aspiration line 324, and the flow through lines 322 and 324 is controlled by the user, for example, via footswitch 326. Power is supplied to handpiece through electrical cable 400.

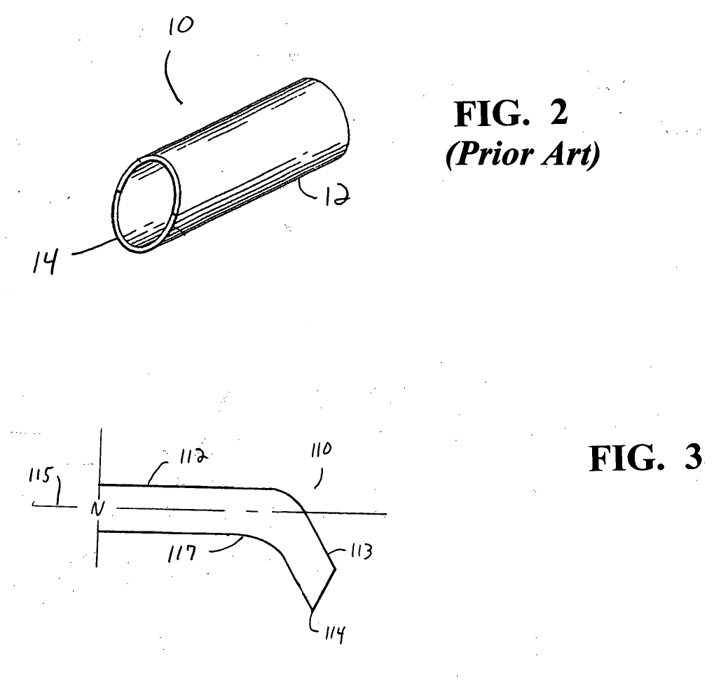

As best seen in FIG. 2, prior art phacoemulsification tip 10 contains shaft 12 that is straight all the way to distal tip 14. As best seen in FIG. 3, phacoemulsification tip 110 which may be used with the method of the present invention contains shaft 112 that is straight up to distal end 113. Distal end 113 is angled or bent on an angle relative to centerline 115 of shaft 112 from intersection 117 of shaft 112 and distal end 113 all the way to distal tip 114.

As best seen in FIG. 3, phacoemulsification tip 110 contains shaft 112 that is straight up to distal end 113. Distal end 1 13 is angled or bent on an angle relative to centerline 115 of shaft 112 from intersection 117 of shaft 112 and distal end 113 all the way to distal tip 114. The inventors have surprisingly discovered that torsional ultrasonic vibration of tip 110 causes a twisting of shaft 112 that is not present if tip 110 is rotarily oscillated. Such twisting causes distal tip 114 to assume a whipping motion which although less that the rotary motion generated in distal tip 114 when tip 110 is rotarily oscillated, the whipping motion greatly increases the cutting efficiency of tip 100.

Cutting tip 110 is preferably made from stainless steel or titanium, but other materials may also be used. Cutting tip 110 preferably has an overall length of between 0.50 inches and 1.50 inches, with 1.20 inches being most preferred. Cutting tip 110 may be formed using conventional metalworking technology and preferably is electropolished to remove any burrs.

Shaft 112 is generally tubular, with an outside diameter of between 0.005 inches and 0.100 inches and an inside diameter of between 0.001 inches and 0.090 inches. Distal end 114 of shaft 112 may be cut square or cut at any suitable angle between 0° and 90°.

This description is given for purposes of illustration and explanation. It will be apparent to those skilled in the relevant art that changes and modifications may be made to the invention described above without departing from its scope or spirit.

We claim:

1. A surgical method, comprising the steps of:
a) inserting a phacoemulsification handpiece having a tip into an eye, the tip having a tubular shaft, the shaft having a distal end that is angled or bent on an angle relative to a centerline of the shaft; and
b) torsionally and ultrasonically vibrating the tip.

2. A surgical method, comprising the steps of:
a) inserting a phacoemulsification handpiece having a tip into an eye, the tip having a tubular shaft, the shaft having a distal end that is angled or bent on an angle relative to a centerline of the shaft; and
b) ultrasonically vibrating the tip so as to produce a twisting motion in the shaft.

3. A surgical method, comprising the steps of:
a) inserting a phacoemulsification handpiece having a tip into an eye, the tip having a tubular shaft, the shaft having a distal end that is angled or bent on an angle relative to a centerline of the shaft; and
b) ultrasonically vibrating the tip so as to produce a whipping motion in the distal end.
